(19) United States
(12) Patent Application Publication
(10) Pub. No.: US 2003/0092608 A1
(43) Pub. Date: May 15, 2003

(54) Pharmaceutical composition for inhibiting the metastasis or preventing the recurrence of malignant tumor

(76) Inventors: Takayuki Kawaguchi, Tokyo-to (JP); Satoshi Okuno, Misato-shi (JP); Toshiro Yano, Suita-shi (JP)

Correspondence Address: BIRCH STEWART KOLASCH & BIRCH, PO BOX 747, FALLS CHURCH, VA, 22040-0747, US

(21) Appl. No.: 10/224,475

(22) Filed: Aug. 21, 2002

Related U.S. Application Data
(60) Provisional application No. 60/331,255, filed on Nov. 13, 2001.

(30) Foreign Application Priority Data
Aug. 21, 2001 (JP) 2001-249717

(51) Int. Cl. A61K 38/14; C07K 9/00; A61K 31/7052

(52) U.S. Cl. 514/8; 530/322; 536/53

(57) ABSTRACT

A pharmaceutical composition for inhibiting the metastasis or preventing the recurrence of a malignant tumor, which comprises as the active ingredient a polysaccharide derivative comprising a polysaccharide having a carboxyl group bound to an active substance having an anti-tumor activity via an amino acid or a peptide consisting of 2 to 8 amino acids which are the same or different, or a salt thereof.

### TECHNICAL FIELD

The present invention relates to a pharmaceutical composition for inhibiting the metastasis or preventing the recurrence of a malignant tumor. More particularly, the present invention relates to a pharmaceutical composition for inhibiting the metastasis or preventing the recurrence of a malignant tumor, which comprises as the active ingredient a polysaccharide derivative comprising a polysaccharide having a carboxyl group bound to an active substance having an anti-tumor activity, for example, a camptothecin derivative of the formula (I) or (II) as mentioned below, via an amino acid or a peptide consisting of 2 to 8 amino acids which are the same or different, or a salt thereof.

### BACKGROUND ART

Malignant tumors are one of the main causes of death in the developed countries, and the majority of malignant tumors related deaths are due to metastasis into distant organs or recurrence accompanied by metastasis to distant organs after a topical therapy. The metastasis to distant organs may be caused by hematogenous metastasis or lymphogenous metastasis, and it is known that a patient having lymphogenous metastasis has a high risk of recurrence of a malignant tumor after topical therapy. The main organs of recurrence are brain, lung, liver, and bone. Especially, a tumor in digestive apparatus, for example, colon cancer from which a large number of patients are suffered, may often invade and spread to the liver, and a breast cancer and a lung cancer as well often invade and spread to the liver. Further, a lymphoma and a lymphatic leukemia may spread mainly to the lymph system, and it has been reported that the metastasis to liver was observed in high rate by autopsy.

In order to inhibit the recurrence including the metastasis to distant organs such a metastasis to the liver and to prolong life, a chemotherapy, etc. is employed as a supportive care after a topical therapy, but the chemotherapy has a potent toxicity and cannot be used for chronic administration. In addition, it has scarcely been reported that the lifetime is more prolonged by a supportive care of chemotherapy than a topical therapy alone. For example, in the trials of post-surgery chemotherapy for the patient who is the subject of surgery of advanced gastric cancer, one of the cancers of digestive organs, clinical tests of various agents for anti-malignant tumors have been tried, but any therapeutic method exhibiting a remarkably better survival rate than a surgery alone has not been established yet.

Under these circumstances, it has been desired to find a new agent effective in inhibiting recurrence or in prolonging life after topical therapy, which is applicable to the lymph node and the distant organs of metastasis with little side effects, and is suitable for chronic administration.

On the other hand, WO 94/19376, WO 97/46260, WO 97/38727, JP-A-10-72467 and JP-A-10-95802 disclose a polysaccharide derivative comprising a polysaccharide bound to an active substance having an anti-tumor activity via an amino acid or a peptide.

However, these publications disclose the use of these polysaccharides in the treatment of cancers by accumulating at the tumor site and killing the tumor cells, but never indicate activities of inhibiting metastasis or preventing recurrence of a malignant tumor.

### DISCLOSURE OF INVENTION

An object of the present invention is to provide a novel pharmaceutical composition for inhibiting the metastasis or preventing the recurrences of a malignant tumor.

The present inventors have intensively studied, and have found that a polysaccharide derivative comprising a polysaccharide having a carboxyl group bound to an active substance having an anti-tumor activity via an amino acid or a peptide exhibits an excellent effect in the inhibition of metastasis and/or prevention of recurrence of a malignant tumor, and have accomplished the present invention. That is, the present invention relates to pharmaceutical composition for inhibiting the metastasis or preventing the recurrence of a malignant tumor, which comprises as the active ingredient a polysaccharide derivative comprising a polysaccharide having a carboxyl group bound to an active substance having an anti-tumor activity via an amino acid or a peptide consisting of 2 to 8 amino acids which are the same or different, or a salt thereof.

### BRIEF DESCRIPTION OF DRAWINGS

FIG. 1 shows the lapsed days after implantation of tumor and the number of survived animals in M 5076 liver metastatic models.

### BEST MODE FOR CARRYING OUT THE INVENTION

The polysaccharide having a carboxyl group of the present invention includes the same ones as those disclosed in the above mentioned WO 94/19376 and WO 97/46260, and includes polysaccharide having originally carboxyl groups in the structure thereof (e.g., hyaluronic acid, pectic acid, alginic acid, chondroitin, heparin, etc.), and polysaccharides having originally no carboxyl group (e.g., pullulan, dextran, mannan, chitin, mannoglucan, chitosan, etc.) but being introduced thereto carboxyl groups, and polysaccharides having originally no carboxyl group in the structure thereof but being introduced thereto carboxyl groups after polyalcohol formation (e.g., polysaccharide polyalcohol having a carboxyl group).

The polysaccharide having originally no carboxyl group but being introduced thereto a carboxyl group means ones that are prepared by substituting a hydrogen atom of a part or all of hydroxyl groups of polysaccharides having originally no carboxyl group with a carboxy-C₁₋₄ alkyl group.

In the present invention, the polysaccharide having a polysaccharide includes one that are prepared by treating a polysaccharide originally having no carboxyl group with a reducing agent, and then followed by substituting a hydrogen atom of a part or all of hydroxyl groups of the resultant with a carboxy-C₁₋₄ alkyl group.

The polysaccharide polyalcohol having a carboxyl group includes, for example, a carboxy-C₁₋₄ alkyl-polysaccharide polyalcohol which is prepared by treating a polysaccharide originally having no carboxyl group successively with sodium periodate and sodium borohydride by the method disclosed in WO 97/46260 to give a polysaccharide polyalcohol, which is further treated with a halogenated C₁₋₄ alkylcarboxylic acid.

The alkyl moiety of the carboxyl-C₁₋₄ alkyl group which substitutes a hydrogen atom of the hydroxyl groups of the above polysaccharide (including a polysaccharide polyalcohol) may be either a straight chain alkyl group or a branched chain alkyl group.

Preferable carboxy-C₁₋₄ alkyl group is, for example, carboxymethyl group, 1-carboxyethyl group, 3-carboxypropyl group, 1-methyl-3-carboxypropyl group, 2-methyl-3-carboxy-propyl group, 4-carboxybutyl group, etc., and carboxymethyl group is more preferable.

In the present invention, the polysaccharide having a carboxyl group is preferably carboxy-C₁₋₄ alkyldextran or carboxy-C₁₋₄ alkyldextran polyalcohol, and carboxyl-C₁₋₄ alkyldextran is especially preferable.

The degree of polyalcohol formation (by the successive oxidation with sodium periodate and reduction with sodium borohydride) in the step of preparing the carboxy-C₁₋₄ alkyl-polysaccharide polyalcohol as mentioned above is not specified, but the intermediate polysaccharide polyalcohol is preferably one being obtained by treating a polysaccharide under possible conditions for substantially almost completely forming polyalcohol.

Moreover, in the present invention, the polysaccharide having a carboxyl group is preferably carboxymethylated dextran or carboxymethylated dextran polyalcohol, and among these polysaccharides, particularly dextran having an average molecular weight of 20,000 to 500,000 is more preferable, and dextran having an average molecular weight of 50,000 to 350,000 is most preferable (said average molecular weight being determined by Gel permeation chromatography (GPC) method, Shinseikagaku, Jikken Koza, vol. 20, p. 7, Tokyo-Kagaku-Dojin, Nov. 5, 1991).

When introducing a carboxylalkyl group into polysaccharides, the degree of the introduction thereof is expressed by “degree of substitution” which is defined by a number of carboxylalkyl groups (including groups of peptide chain being introduced by these groups) per a sugar residue. That is expressed by the following equation.

$$\begin{matrix}\text{Degree of} \\ \text{Substitution}\end{matrix} = \frac{\begin{matrix}\text{Number of carboxyalkyl groups} \\ \text{in the molecule}\end{matrix}}{\begin{matrix}\text{Total number of} \\ \text{sugar residues in the molecule}\end{matrix}}$$

When the carboxylalkyl group is carboxymethyl group, the degree of substitution is occasionally expressed by the degree of carboxymethylation (CM-degree).

When the polysaccharide is dextran, the degree of substitution thereof is preferably in the range of 0.3 to 0.8. When the polysaccharide is dextran polyalcohol, the degree of substitution is preferably in the range of 0.3 to 0.5.

The amino acid or peptide of the present invention plays a role of spacer existing between a polysaccharide having a carboxyl group and an active substance having an anti-tumor activity, and the amino acid or amino acid forming said peptide includes both natural amino acids and synthetic amino acid (including D-amino acids, L-amino acids, a mixture thereof), and also includes either neutral amino acids, basic amino acids or acidic amino acids. Moreover, the amino acid of the present invention may be not only α-amino acid but also β-amino acids, γ-amino acids, ε-amino acids, etc.

Examples of the amino acids are glycine, α-alanine, β-alanine, valine, leucine, isoleucine, serine, threonine, systeine, methionine, aspartic acid, glutamic acid, lysine, citrulline, arginine, phenylalanine, tyrosine, histidine, tryptophan, proline, hydroxyproline, γ-aminobutyric acid, ε-aminocaproic acid, etc.

The peptide of the present invention includes ones consisting of 2 to 8 amino acids, preferably 2 to 5 amino acids, which are the same or different. Examples of the peptides are glycyl-glycyl-L- or D-phenylalanyl-glycine, glycyl-glycine, glycyl-glycyl-glycine, glycyl-glycyl-glycyl-glycine, glycyl-glycyl-glycyl-glycyl-glycine, L- or D-phenylalanyl-glycine, L- or D-tyrosyl-glycine, L- or D-leucyl-glycine, L- or D-phenylalanyl-citrulline and L- or D-valyl-citrullin (the N-terminus of these peptides is introduced onto the carboxyl group of a polysaccharide).

Among these peptides, glycyl-glycyl-L- or D-phenyl-alanyl-glycine, glycyl-glycine, glycyl-glycyl-glycine, glycyl-glycyl-glycyl-glycine, glycyl-glycyl-glycyl-glycyl-glycine, and L- or D-phenylalanyl-glycine are preferable.

The active substance having an anti-tumor activity of the present invention may include various compounds being known as an anti-tumor agent, and may be either cytotoxic agents or cytostatic agents. The cytotoxic agent is preferably camptothecin derivatives and taxane derivatives, and the cytostatic agent is preferably angiogenesis inhibitors, EGF receptor inhibitors. More preferably, the cytotoxic agent is camptothecin derivatives, and the cytostatic agent is angiogenesis inhibitors.

Examples of camptothecin derivatives are compounds disclosed in JP-A-10-72467 of the formula (I):

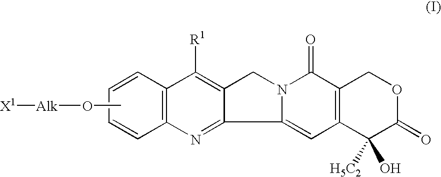

wherein R¹ is a substituted or unsubstituted lower alkyl group, X¹ is a group of the formula: —NHR² (R² is a hydrogen atom or a lower alkyl group) and Alk is a straight chain or branched chain C₁₋₆ alkylene group having optionally an oxygen atom in the chain thereof. Among them, preferable compound is 10-(3′-aminopropyloxy)-7-ethyl-(20S)-camptothecin.

Other examples of camptothecin derivatives are compounds disclosed in JP-A-10-95802 of the formula (II):

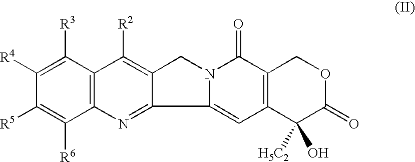

wherein two groups of R² to R⁶ being adjacent each other combine to form a lower alkylene group, and one of the carbon atoms of said lower alkylene group is substituted by an amino group, and the remaining three groups of R² to R⁶ are a hydrogen atom, a lower alkyl group or a halogen atom. Among them, preferable compound is (1S, 9S)-1-amino-9-ethyl-5-fluoro-2,3-dihydro-9-hydroxy-4-methyl-1H,12H-benzo[de]pyrano[3′,4′:6,7]indolidino[1,2-b]quinoline-10,13(9H,15H)-dione, etc.

Examples of taxane derivatives are Taxol, Taxotere, 13-[(2′R,3′R)-3′N-t-butyloxycarbonyl-3′-cyclopropyl]-10-deacetyl-baccatin III, etc.

In the active ingredient of the present invention, the ratio of the polysaccharide and the active substance having an anti-tumor activity may be selected according to the kinds of the polysaccharide to be used, but when the polysaccharide is dextran or dextran polyalcohol, then the content of the active substance having an anti-tumor activity is preferable in the range of 0.1 to 20% by weight, more preferably in the range of 2 to 10% by weight, based on the whole weight of the active ingredient.

Among the active ingredients of the present invention, preferable ones are polysaccharide derivatives or a salt thereof wherein an amino acid or a peptide consisting of 2 to 8 amino acids which are the same or different are introduced into a part or all of the carboxyl groups of the polysaccharide having a carboxyl group through an acid-amide bond, and the remaining part or all of the amino groups or carboxyl groups which do not participate in the binding to the carboxyl groups of the above peptide are bound to the carboxyl groups, amino groups or hydroxyl groups of the active substance having an anti-tumor activity through an acid-amide bond or ester bond.

Especially preferable active ingredient is a polysaccharide derivative, wherein the polysaccharide having an carboxyl group is carboxymethylated dextran, the active substance having an anti-tumor activity is 10-(3′-aminopropyloxy)-7-ethyl-(20S)-camptothecin, and the peptide is glycyl-glycyl-glycine, or a salt thereof. Especially preferable one is a polysaccharide derivative wherein the polysaccharide having a carboxyl group is carboxymethylated dextran having an average molecular weight of 60,000 to 200,000, and the degree of carboxymethylation thereof is in the range of 0.3 to 0.8, or a salt thereof.

Other preferable active ingredient is a polysaccharide derivative wherein the polysaccharide having a carboxyl group is a carboxy-C₁₋₄ alkyldextran polyalcohol, the active substance having an anti-tumor activity is (1S, 9S)-1-amino-9-ethyl-5-fluoro-2,3-dihydro-9-hydroxy-4-methyl-1H,12H-benzo[de]pyrano[3′,4′:6,7]indolidino[1,2-b]-quinoline-10,13(9H,15H)-dione, and the peptide is glycyl-glycyl-L- or D-phenylalanyl-glycine, or a salt thereof, and especially preferable ones are a polysaccharide derivative or a salt thereof wherein the polysaccharide having a carboxyl group is carboxy-C₁₋₄ alkyldextran polyalcohol having an average molecular weight of 200,000 to 400,000, and the degree of the substitution thereof is in the range of 0.3 to 0.5.

The polysaccharide derivative or a salt thereof of the active ingredient of the present invention may be prepared according to the methods disclosed in WO 94/19376, WO 97/46260, WO 97/38727, JP-A-10-72467, and JP-A-10-95802.

The pharmaceutical composition of the present invention may highly accumulate at the site such as lymph node or the liver to which cancers may spread, can release an active substance at an appropriate rate so that the active substance hardly affects on the normal cells and suppressively acts on the growth of tumor cells, and hence, the pharmaceutical composition of the present invention is useful in the inhibition of metastasis or prevention of reoccurrence of a malignant tumor. Especially, the pharmaceutical composition of the present invention is useful in the inhibition of lymph node metastasis or liver metastasis, particularly useful in the inhibition of lymph node metastasis. Further, among lymph node metastasis, the present pharmaceutical composition is useful in the inhibition of metastasis in lymph node from the colon, or metastasis in lymph node from the lung.

In addition, the present pharmaceutical composition may exhibit its effects not only before the onset of metastasis but also after the onset of metastasis. Therefore, the present pharmaceutical composition is also useful in the inhibition of metastasis or prevention of reoccurrence of a malignant tumor after a topical therapy (e.g., surgery, radiation therapy, thermotherapy, cryotherapy, laser burning therapy, etc.). Moreover, the present pharmaceutical composition is also suitable for repetitive dosing for long time, and can be employed together with a topical therapy.

The present pharmaceutical composition is preferably administered parenterally (e.g., intravenous injection), and is usually administered in the form of a liquid preparation such as solution, suspension, emulsion, etc.

The present pharmaceutical composition is preferably formulated in the form of an injection or drip infusion by using distilled water for injection, physiological saline solution, aqueous glucose solution.

The dosage of the present pharmaceutical composition may vary according to the administration methods, age, weight or conditions of the patients, etc., but it is usually in the range of 0.002 to 50 mg/kg, more preferably in the range of 0.01 to 5 mg/kg, in single dose, converted into an amount of the active substance.

In the present specification, the lower alkyl group and the lower alkylene group may be ones having 1 to 6 carbon atoms, preferably ones having 1 to 4 carbon atoms, and the halogen atom is fluorine atom, chlorine atom, bromine atom, iodine atom, etc.

EXPERIMENTS

Experiment 1 (M 5076 Liver Metastatic Models)

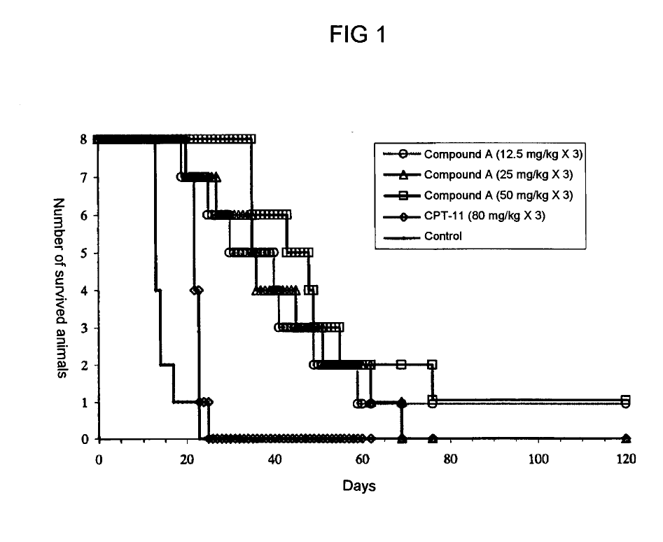

One million of M 5076 cells (mouse ovarian sarcoma cells) were implanted into BDF1 male mice (5-weeks old, 8 animals per group) at the tail vein. A test compound (Compound A; the compound obtained in Preparation 1 as described below and Irinotecan (CPT-11)) was dissolved in a physiological saline solution, and each amount as indicated in Table 1 as mentioned below was administered intravenously to the mice on the 4th, 8th and 12th day after the implantation, and the mice were observed for 120 days after the implantation of tumor. In the control group (untreated with test compound), only a physiological saline solution was administered. The survival time (days) was measured in both the test compound-treated groups and the control group, and the prolongation rate of survival was calculated according to the following equation. The results are shown in Table 1 and FIG. 1.

TABLE 1
$\begin{matrix}Prolongation rate \\ of survival\end{matrix} = ( \frac{\begin{matrix}Survival days in the test \\ compound \text{-} treated group\end{matrix}}{\begin{matrix}Survival days in \\ the control group\end{matrix}} - 1 ) \times 100$

| | | | | Prolon- |
|---|---|---|---|---|
| | | | | gation |
| | | | | rate of |
| | Dose | Survival | Standard | survival |
| | (mg/kg) | days | error | (%) |
| Control | | 15.00 | 1.24 | — |
| Compound A | 12.5 | 37.57 | 5.27 | 150.5 |
| | 25 | 43.13 | 5.98 | 187.5 |
| | 50 | 48.71 | 5.33 | 224.8 |
| Irinotecan | 80 | 22.50 | 0.5 | 50.0 |

As is shown in Table 1, the compound obtained in Preparation 1 as mentioned below (Compound A) exhibited an excellent activity of prolonging lifetime in M5076 liver metastatic models. Meanwhile, Irinotecan is not known as an agent for inhibiting the metastasis or preventing the reoccurrence of a malignant tumor, but it was merely tested as a drug of camptothecin derivatives.

Experiment 2 (HT-29 Metastatic Models)

A segment (2 mm²) of HT-29 cells (human colon cancer) was implanted into the vermiform appendix of 100 NCr nu/nu female mice (5 to 6 weeks old, 10 animals per group). A test compound (Compound A; the compound obtained in Preparation 1 as mentioned below, Compound B; the compound obtained in Preparation 4 as mentioned below, and Irinotechan (CPT-11)) was dissolved in a physiological saline solution, and each amount as indicated in Table 2 as mentioned below was administered intravenously to the mice on the 15th, 19th, 23rd and 25th day after the implantation of tumor. On the other hand, in the control group (untreated with test compound), only a physiological saline solution was administered. The presence or absence of the metastasis of each organ was checked on the 84th day after the implantation of tumor. The results are shown in the following Table 2.

TABLE 2

| | | | | | | | | | | Number of | |
|---|---|---|---|---|---|---|---|---|---|---|---|
| | number | | | | | | | Other | | animals with | |
| | of | Lymph node | | Liver | | Lung | | organs*** | | metastasis | |
| Group | animals | MI* | P** | MI* | P** | MI* | P** | MI* | P** | | P** |
| Compound A | 10 | 0 | <0.01 | 0 | 1.0 | 0 | 0.21 | 0 | 1.0 | 0 | <0.01 |
| (40 mg/kg)**** | | | | | | | | | | | |
| Compound A | | | | | | | | | | | |
| (20 mg/kg)**** | 10 | 1 | <0.01 | 0 | 1.0 | 1 | 0.58 | 0 | 1.0 | 2 | <0.01 |
| Compound A | | | | | | | | | | | |
| (10 mg/kg)**** | 10 | 8 | 1.0 | 0 | 1.0 | 2 | 1.0 | 0 | 1.0 | 8 | 1.0 |
| Compound A | 10 | 6 | 0.3 | 0 | 1.0 | 1 | 0.58 | 2 | 1.0 | 6 | 0.30 |
| (5 mg/kg)**** | | | | | | | | | | | |
| Compound B | | | | | | | | | | | |
| (5 mg/kg)***** | 10 | 0 | <0.01 | 0 | 1.0 | 0 | 0.21 | 0 | 1.0 | 0 | <0.01 |
| Compound B | | | | | | | | | | | |
| (2.5 mg/kg)***** | 10 | 6 | 0.30 | 1 | 1.0 | 1 | 0.58 | 1 | 1.0 | 6 | 0.3 |
| Irinotecan | 10 | 7 | 0.58 | 0 | 1.0 | 1 | 0.58 | 1 | 1.0 | 7 | 0.58 |
| (40 mg/kg) | | | | | | | | | | | |
| Irinotecan | 10 | 9 | 1.0 | 2 | 1.0 | 2 | 1.0 | 0 | 1.0 | 9 | 1.0 |
| (20 mg/kg) | | | | | | | | | | | |
| Control | 10 | 9 | — | 1 | — | 3 | — | 1 | — | 9 | — |

*MI means Metastatic Incidence
**P means standard derivation, where all treated groups compared to Control by Fischer exact test.
***Including Diaphragm, Abdominal cavity and Thoracic cavity.
****Dosage converted into 10-(3′-aminopropyloxy)-7-ethyl-(20S)-camptothecin
*****Dosage converted into (1S,9S)-1-amino-9-ethyl-5-fluoro-2,3-dihydro-9-hydroxy-4-methyl-1H,12H-benzo[de]pyrano[3′,4′:6,7]indolidino[1,2-b]quinoline-10,13(9H,15H)-dione

Experiment 3 (HT-29 Metastatic Models)

A segment (2 mm²) of HT-29 cells (human colon cancer) was implanted into the vermiform appendix of 100 NCr nu/nu female mice (5 to 6 weeks old, 10 animals per group). Since the lymph node metastasis was observed on the 49th day after the implantation of tumor, a test compound (Compound A; the compound obtained in Preparation 1 as mentioned below, and Irinotechan (CPT-11)) was dissolved in a physiological saline solution, and each amount as indicated in Table 3 as mentioned below was administered intravenously to the mice on the 51st, 55th, 59th, and 63rd day after the implantation of tumor. On the other hand, in the control group (untreated with test compound), only a physiological saline solution was administered. The presence or absence of the metastasis of each organ was checked on the 84th day after the implantation of tumor. The results are shown in the following Table 3.

TABLE 3

| | | | | | | | | | | Number of | |
|---|---|---|---|---|---|---|---|---|---|---|---|
| | Number | | | | | | | Other | | animals with | |
| | of | Lymph node | | Liver | | Lung | | organs*** | | metastasis | |
| Group | animals | MI* | P** | MI* | P** | MI* | P** | MI* | P** | | P** |
| Compound A | 10 | 0 | <0.01 | 0 | 1.0 | 0 | 0.21 | 2 | 1.0 | 2 | <0.01 |
| (40 mg/kg)**** | | | | | | | | | | | |
| Compound A | | | | | | | | | | | |
| (20 mg/kg)**** | 10 | 2 | <0.01 | 0 | 1.0 | 0 | 0.21 | 1 | 1.0 | 3 | <0.01 |
| Irinotecan | 10 | 8 | 1.0 | 1 | 1.0 | 0 | 0.21 | 1 | 1.0 | 8 | 1.0 |
| (40 mg/kg) | | | | | | | | | | | |
| Control | 10 | 9 | — | 1 | — | 3 | — | 1 | — | 9 | — |

*MI means Metastatic Incidence
**P means standard derivation, where all treated groups compared to Control by Fischer exact test.
***Including Diaphragm, Abdominal cavity and Thoracic cavity.
****Dosage converted into 10-(3′-aminopropyloxy)-7-ethyl-(20S)-camptothecin

Experiment 4 (H460 Metastatic Models)

A segment (2 mm²) of H460 cells (human lung cancer) was implanted into the left lung of 100 NCr nu/nu female mice (5 to 6 weeks old, 10 animals per group). Since the metastasis was observed on the 14th day after the implantation of tumor in another control group, a test compound (Compound A; the compound obtained in Preparation 1 as mentioned below, Compound B; the compound obtained in Preparation 4 as mentioned below, and Irinotechan (CPT-11)) was dissolved in a physiological saline solution, and each amount as indicated in Table 4 as mentioned below was administered intravenously to the mice on the 14th, 18th, 22nd and 26th day after the implantation of tumor. On the other hand, in the control group (untreated with test compound), only a physiological saline solution was administered. The presence or absence of the metastasis of each organ was checked on the 36th day after the implantation of tumor. The results are shown in the following Table 4.

TABLE 4

| | | | | | | | | Number of | |
|---|---|---|---|---|---|---|---|---|---|
| | number | | | | | | | animals with | |
| | of | Lymph node | | Liver | | Lung | | metastasis | |
| Group | animals | MI* | P** | MI* | P** | MI* | P** | | P** |
| Compound A | 10 | 1 | <0.01 | 0 | 1.0 | 0 | 0.54 | 1 | <0.01 |
| (40 mg/kg)**** | | | | | | | | | |
| Compound A | 10 | 2 | 0.05 | 0 | 1.0 | 2 | 0.58 | 3 | 0.245 |
| (20 mg/kg)**** | | | | | | | | | |
| Compound A | 10 | 1 | <0.01 | 0 | 1.0 | 0 | 0.54 | 1 | 0.02 |
| (10 mg/kg)**** | | | | | | | | | |
| Compound A | 10 | 2 | 0.05 | 0 | 1.0 | 1 | 1.0 | 2 | 0.06 |
| (5 mg/kg)**** | | | | | | | | | |
| Compound B | 10 | 2 | 0.05 | 0 | 1.0 | 1 | 1.0 | 2 | 0.06 |
| (5 mg/kg)***** | | | | | | | | | |
| Compound B | 10 | 1 | <0.01 | 0 | 1.0 | 0 | 0.54 | 1 | 0.02 |
| (2.5 mg/kg)***** | | | | | | | | | |
| Irinotecan | 10 | 0 | <0.01 | 0 | 1.0 | 1 | 1.0 | 1 | 0.02 |
| (40 mg/kg) | | | | | | | | | |
| Irinotecan | 10 | 2 | 0.05 | 0 | 1.0 | 0 | 0.54 | 2 | 0.06 |
| (80 mg/kg) | | | | | | | | | |
| Control | 20 | 13 | — | 0 | — | 2 | — | 12 | |

*MI means Metastatic Incidence
**P means standard derivation, where all treated groups compared to Control by Fischer exact test.
****Dosage converted into 10-(3′-aminopropyloxy)-7-ethyl-(20S)-camptothecin
*****Dosage converted into (1S, 9S)-1-amino-9-ethyl-5-fluoro-2,3-dihydro-9-hydroxy-4-methyl-1H,12H-benzo[de]pyrano[3′,4′:6,7]indolidino[1,2-b]quinoline-10,13(9H,15H)-dione

PREPARATIONS

Preparation 1

Preparation of CM-dextran-7-ethyl-10-[3′-(glycyl-glycyl-glycylamino)propyloxy]-(20S)-camptothecin:

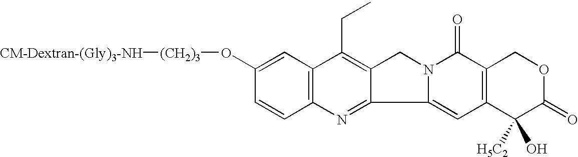

(CM-Dextran means carboxymethyldextran, hereinafter, the same)

(1) 10-(3′-Aminopropyloxy)-7-ethyl-(20S)-camptothecin hydrochloride (500 mg) was dissolved in acetonitrile (25 ml), and thereto were successively added t-butoxycarbonyl glycyl-glycyl-glycine (345 mg), N-methylmorpholine (121 mg), N-hydroxybenzotriazole (161 mg) and 1-(3-dimethylamino-propyl)-3-ethylcarbodiimide hydrochloride (228 mg), and the mixture was stirred overnight. The precipitated product was collected by filtration, purified by silica gel column chromatography to give pale yellow foamy powder, which was recrystallized from n-propanol to give 7-ethyl-10-[3′-(t-butoxycarbonyl-glycyl-glycyl-glycylamino)propyloxy]-(20S)-camptothecin (663 mg) as colorless crystals. M.p.: 157-159° C.

(2) 7-Ethyl-10-[3′-(t-butoxycarbonyl-glycyl-glycyl-glycylamino)propyloxy]-(20S)-camptothecin (3.86 g) was emulsified in purified water (64 ml), and thereto was added 6N aqueous hydrochloric acid solution (32 ml), and the mixture was reacted at room temperature under stirring for 2 hours. The solvent was concentrated, and thereto was added n-propanol to precipitate powdery product. The resulting powdery product was collected by filtration, and recrystallized from aqueous n-propanol to give 7-ethyl-10-[3′-(glycyl-glycyl-glycylamino)propyloxy]-(20S)-camptothecin hydrochloride (2.56 g) as yellow crystals.

(3) CM-Dextran sodium salt (CM-degree=0.44, 50 g) was dissolved in water (2.5 liters), and the pH value thereof was adjusted to pH 5.0 with 0.2N aqueous hydrochloric acid solution under stirring at 15° C., and thereto was added 7-ethyl-10-[3′-(glycyl-glycyl-glycylamino)propyloxy]-(20S)-camptothecin hydrochloride (4.01 g). To the mixture was added 1-(3-dimethylaminopropyl)-3-ethylcarbodiimide hydrochloride (50 g), during which the pH value of the reaction solution was kept at 5.0-5.5 with 0.2N hydrochloric acid. The mixture was reacted at 15° C. under stirred for one hour, and diluted to the total volume of 10 liters with purified water. While the pH value was kept at over pH 4.0, the low molecule fractions were removed by using an ultrafiltration module (ACP-1010, manufactured by Asahi Kasei Industries, Ltd.), and the pH value thereof was adjusted to pH 8 with 0.1N aqueous sodium hydroxide solution, and then subjected to ion-exchange resin MSC-1 (Na-type, manufactured by Dowex). The fractions containing the desired compounds are concentrated, and filtered through a filter (0.45 μm). The resultant was mixed with ethanol (10 liters) with stirring, and thereto was added dropwise 3M brine (40 ml) under stirring. The resulting precipitates were collected by filtration, and dissolved in purified water (21 liters). The pH value of the solution was adjusted to pH 4.0 with 0.2N aqueous hydrochloric acid solution, and subjected again to ultrafiltration during which the pH value was kept at pH 4.0. The solvent was concentrated to the total volume of 1.5 liter, and filtered through a filter (0.45 μm). The resultant was mixed with ethanol (9 liters), and thereto was added dropwise 3M brine (35 ml) under stirring. The resulting precipitates were collected by filtration, and washed successively with ethanol and acetone, concentrated under reduced pressure to give the desired compound (54.9 g) as pale yellow powder. The content as 10-(3′-aminopropyloxy)-7-ethyl-(20S)-camptothecin hydrochloride was confirmed as 4.2% by absorption at 367.5 nm. According to the analysis by GPC (Gel Permeation Chromatograph), the average molecular weight of the desired product was 121 kDa, and the degree of distribution (Mw/Mn) was 1.47.

Preparation 2

Preparation of CM-dextran-13-[(2′R,3′S)-3′-N-tert-butoxycarbonyl-3′-phenyl-2′-O-L-phenylalanyl-glycyl-isoserinyl]-10-deacyl-baccatin III:

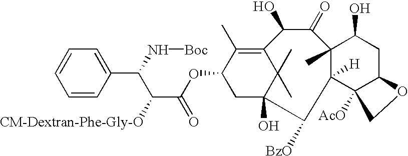

(Bz is benzoyl group, hereinafter, the same)

CM-Dextran(2008 mg, CM-degree: 0.47, the average molecular weight: 170 kDa) was dissolved with stirring in purified water (90 ml), and thereto were added 13-[(2′R,3′S)-3′-N-tert-butoxycarbonyl-3′-phenyl-2′-O-L-phenylalanyl-glycyl-isoserinyl]-10-deacyl-baccatin III mesylate (119 mg) and dimethylformamide (90 ml), and the mixture was stirred so as to dissolve. To the mixture was added with stirring 2-ethoxy-1(2H)-quinolinecarboxylic acid (4.0 g), and the mixture was stirred at room temperature overnight. To the reaction solution was added ethanol (720 ml) with stirring, and thereto was further added dropwise 3M brine (1.8 ml) under stirring. The precipitates were collected by centrifugation, and dissolved in water (200 ml), and the pH value of the solution was adjusted to pH 7 with 0.2N aqueous sodium hydroxide solution. The solution was poured into ethanol (800 ml) with stirring, and thereto was added dropwise 3M brine (4 ml) with stirring. The resulting precipitates were collected by centrifugation, and purified in the same manner as in Preparation 1-(3) to give the desired compound (600 mg) as white powder. The content of the active substance: 2.4% (UV method, (λ=276 nm))

Preparation 3

Preparation of CM-dextran-2′-O-phenylalanyl-glycyl-taxol:

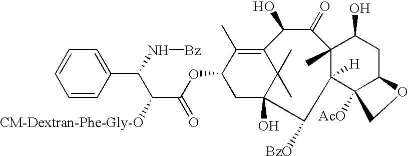

CM-Dextran (1.294 g, CM-degree: 0.47, the average molecular weight: 170 kDa) was dissolved with stirring in purified water (70 ml), and thereto were added 2′-O-phenyl-alanyl-glycyltaxol mesylate (77 mg) and dimethylformamide (70 ml), and the mixture was further stirred so as to dissolve. 2-Ethoxy-1(2H)-quinolinecarboxylic acid (2.59 g) was added to the mixture under stirring, and the mixture was reacted with stirring overnight. The reaction solution was added to ethanol (700 ml) under stirring, and thereto was added dropwise 3M brine (1.4 ml) under stirring. The precipitates were collected by centrifugation, and dissolved in water (240 ml), and mixed with ethanol (1200 ml) under stirring. 3M Brine (4.8 ml) was added drowpise to the mixture under stirring for precipitation. In the same manner, the precipitation was further repeated three times to give the desired product (746 mg) as white powder. The content of the active substance: 4.8% (UV method (λ=273 nm))

Preparation 4

Preparation of carboxymethyldextran-polyalcohol-(1S,9S)-1-(glycyl-glycyl-L-phenylalanyl-glycylamino)-9-ethyl-5-fluoro-2,3-dihydro-9-hydroxy-4-methyl-1H,12H-benzo[de]-pyrano[3′,4′:6,7]indoridino[1,2-b]quinoline-10,13(9H,15H)-dione:

(1) Preparation of (1S,9S)-1-(t-butoxycarbonyl-glycyl-glycyl-L-phenylalanyl-glycylamino)-9-ethyl-5-fluoro-2,3-dihydro-9-hydroxy-4-methyl-1H,12H-benzo[de]pyrano-[3′,4′:6,7]indolidino[1,2-b)quinoline-10,13(9H,15H)-dione:

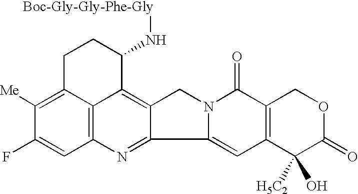

To a solution of (1S,9S)-1-amino-9-ethyl-5-fluoro-2,3-dihydro-9-hydroxy-4-methyl-1H,12H-benzo[de]pyrano-[3′,4′:6,7]indolidino[1,2-b]quinoline-10,13(9H,15H)-dione hydrochloride (167 mg; 0.354 mmol), t-butoxycarbonyl-glycyl-glycyl-L-phenylalanyl-glycine (463 mg; 1.06 mmol) and 1-hydroxybenzotriazole monohydrate (HOBT) (143 mg; 1.06 mmol) in dimethylformamide (DMF) (10 ml) were added 1-(3-dimethylaminopropyl)-3-ethylcarbodimimide (EDC) hydrochloride (270 mg; 1.42 mmol), triethylamine (148 μl; 1.06 mmol) and 4-dimethylaminopyridine (DMAP) (5 mg; 0.04 mmol). The reaction mixture was stirred at room temperature for 15 hours, and the solvent was concentrated under reduced pressure. The residue was dissolved in chloroform, and the mixture was washed, dried, and the solvent was evaporated under reduced pressure. The residue was purified by silica gel column chromatography (solvent; chloroform : methanol=50:1 to 10:1) to give the title compound (228 mg, yield: 75%) as pale yellow solid.

IR (Nujol); 3290, 1710, 1655 cm⁻¹

ESI-MS; 854 (M+H)

(2) Preparation of (1S,9S)-1-(glycyl-glycyl-L-phenyl-alanyl-glycylamino)-9-ethyl-5-fluoro-2,3-dihydro-9-hydroxy-4-methyl-1H,12H-benzo[de]pyrano[3′,4′:6,7]indolidino[1,2-b]quinoline-10,13(9H,15H)-dione:

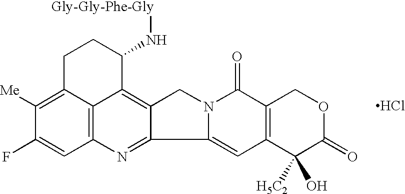

To a solution of (1S,9S)-l-(t-butoxycarbonyl-glycyl-glycyl-L-phenylalanyl-glycylamino)-9-ethyl-5-fluoro-2,3-dihydro-9-hydroxy-4-methyl-1H,12H-benzo[de]pyrano-[3′,4′:6,7]indolidino[1,2-b]quinoline-10,13(9H,15H)-dione (220 mg; 0.258 mmol) in dioxane (4 ml) was added 4N hydrogen chloride solution in dioxane (6 ml) under stirring in an ice bath. The mixture was stirred at room temperature for 16 hours. Diethyl ether (30 ml) was added to the reaction mixture, and the mixture was stirred at room temperature for one hour. The precipitates were collected by filtration, and dried to give the title compound (176 mg, yield; 86%) as yellow powder.

IR (Nujol); 3250, 1745, 1660, 1605, 1535 cm⁻¹

ESI-MS; 754 (M+H)

(3) Preparation of dextran polyalcohol (PA-Dextran):

Acetic buffer (0.1 M, pH 5.5, 1000 ml) was put into a three-neck round bottom flask (capacity; 3 liters). Dextran T-500® (10.0 g, manufactured by Amersham Pharmacia Biotech AB) was added in small portions to the buffer over a period of 30 minutes at room temperature. The mixture was stirred for about 30 minutes until the solution became clear, and then, the mixture was cooled at 5° C. (inner temperature) in a bath.

Separately, to a flask (capacity; 1 liter) were added sodium periodate (33.0 g) and water (1000 ml), and the mixture was stirred at room temperature, and then cooled at 5° C.

To the above dextran solution was added with stirring the above sodium periodate solution at 5° C., and the mixture was ketp at 5° C. for 5 days in a dark place. The excess sodium periodate was removed by adding ethylene glycol (10 ml), and the mixture was further stirred at 5° C. for 2 hours. The reaction mixture was cooled to 3° C., and thereto was added 8M aqueous sodium hydroxide solution during which the reaction temperature was kept below 6° C. (the pH value of the reaction mixture became over pH 9). To the reaction mixture was added sodium borohydride (14 g) in small portions with stirring, and the mixture was stirred at 5° C. overnight. In order to remove the excess sodium borohydride, the pH value of the reaction mixture was adjusted to below pH 5.5 by adding acetic acid thereto at 3 to 6° C., and the mixture was further stirred for 2 hours. The pH value of the reaction mixture was adjusted to about pH 7.8 with 8M aqueous sodium hydroxide solution. The mixture was subjected to dialysis against water (Spectora®/Por 3 membrane, Molecule weight cutoff<3500), and lyophilized to give dextran polyalcohol (8.34 g) as amorphous powder.

(4) Preparation of carboxymethyldextran polyalcohol (CM-PA-Dextran):

Water (155 ml) was put into a three-neck round bottom flask (capacity; 500 ml), and thereto was added with stirring dextran polyalcohol (5.18 g) at room temperature over a period of 10 minutes. The mixture was stirred for about 10 to 30 minutes until the mixture became clear, and then sodium hydroxide (pellet, 97.0%, 21.8 g) was added to the dextran polyalcohol solution in small portions under stirring, during which the inner temperature was kept at 30 to 40° C. in an ice bath. The reaction flask was put in a bath, and the mixture was stirred at 30° C. Chloroacetic acid (31.1 g) was added with stirring in small portions into the reaction mixture at 30 to 40° C. After the addition, the mixture was further stirred at 30° C. in a bath for 20 hours. The reaction mixture was cooled in an ice bath, and the mixture was neutralized by adding thereto acetic acid under stirring (i.e., the pH value was adjusted to below pH 9).

Water (160 ml) was added to the mixture, and the mixture was subjected to dialysis against water (Spectora®/Por 3 membrane, Molecule weight cutoff<3500), and lyophilized to give carboxymethydextran polyalcohol (6.53 g) as amorphous powder.

(5) Preparation of carboxymethyldextran-polyalcohol-(1S,9S)-1-(glycyl-glycyl-L-phenylalanyl-glycylamino)-9-ethyl-5-fluoro-2,3-dihydro-9-hydroxy-4-methyl-1H,12H-benzo[de]pyrano[3′,4′:6,7]indolidino[1,2-b]quinoline-10,13(9H,15H)-dione:

Water(40 ml) was put into a round bottom flask (capacity; 100 ml), and thereto was added carboxymethyldextran polyalcohol (1.0 g) at room temperature with stirring over a period of 5 minutes. The mixture was stirred about 30 minutes until the mixture became clear. A solution of (1S,9S)-1-(glycyl-glycyl-L-phenylalanyl-glycylamino)-9-ethyl-5-fluoro-2,3-dihydro-9-hydroxy-4-methyl-1H,12H-benzo[de]pyrano[3′,4′:6,7]indolidino[1,2-b]quinoline-10,13(9H,15H)-dione in dimethylformamide (100 mg/10 ml) was added with stirring to the mixture, and further added thereto dimethylformamide (15 ml), and the mixture was stirred for 10 minutes. To the mixture was added dropwise with stirring a solution of 2-ethoxy-1-ethoxycarbonyl-1,2-dihydroquinoline (EEDQ) in dimethyl-formamide (1.0 g/10 ml) at room temperature, and the mixture was further stirred for 18 hours. The reaction mixture was subjected to dialysis against water (Spectora®/Por 3 membrane, Molecule weight cutoff<3500), and further purified by cation exchange column (BioRad AG® MP-50 column, Na-type, 30 ml). The main fraction was subjected to dialysis (Spectora®/Por 3 membrane, Molecule weight cutoff<3500), and lyophilized to give a crude product, which was pulverized with acetone, collected by filtration, and dried to give the desired product (904 mg) as pale yellow powder.

INDUSTRIAL APPLICABILITY

The pharmaceutical composition of the present invention may highly accumulate at the site such as lymph node or the liver to which cancers may spread, and suppressively act on the growth of tumor cells without affecting on the normal cells, and hence, the pharmaceutical composition of the present invention is useful in the inhibition of metastasis, particularly in the inhibition of lymph node metastasis or liver metastasis, or prevention of reoccurrence of a malignant tumor.

In addition, the present pharmaceutical composition may exhibit its effects not only before the onset of metastasis but also after the onset of metastasis. Therefore, the present pharmaceutical composition is also useful in the inhibition of metastasis or prevention of reoccurrence of a malignant tumor after topical therapy (e.g., surgery, radiation therapy, thermotherapy, cryotherapy, laser burning therapy, etc.).

1. A pharmaceutical composition for inhibiting the metastasis or preventing the recurrence of a malignant tumor, which comprises as the active ingredient a polysaccharide derivative comprising a polysaccharide having a carboxyl group bound to an active substance having an anti-tumor activity via an amino acid or a peptide consisting of 2 to 8 amino acids which are the same or different, or a salt thereof.

2. The pharmaceutical composition according to claim 1, wherein the active substance having an anti-tumor activity is a camptothecin derivative of the formula (I): 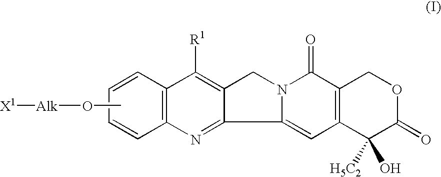
wherein R¹ is a substituted or unsubstituted lower alkyl group, X¹ is a group of the formula: —NHR² (R² is a hydrogen atom or a lower alkyl group) and Alk is a straight chain or branched chain C₁₋₆ alkylene group having optionally an oxygen atom in the chain thereof, or a compound of the formula (II): 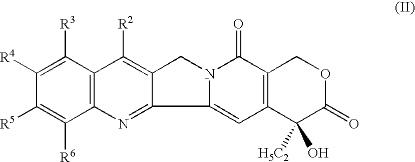
wherein two groups of R² to R⁶ being adjacent each other combine to form a lower alkylene group, and one of the carbon atoms of said lower alkylene group is substituted by an amino group, and the remaining three groups of R² to R⁶ are a hydrogen atom, a lower alkyl group or a halogen atom.

3. The pharmaceutical composition according to claim 1 or claim 2, wherein the polysaccharide having a carboxyl group is a carboxy-C₁₋₄ alkyldextran or a carboxy-C₁₋₄ alkyldextran polyalcohol.

4. The pharmaceutical composition according to claim 1 or claim 2, wherein the polysaccharide having a carboxyl group is a carboxy-C₁₋₄ alkyldextran.

5. The pharmaceutical composition according to any one of claims 1, 2, 3 and 4, wherein the peptide is a member selected from the group consisting of glycyl-glycyl-L- or D-phenylalanyl-glycine, glycyl-glycine, glycyl-glycyl-glycine, glycyl-glycyl-glycyl-glycine, glycyl-glycyl-glycyl-glycyl-glycine, L- or D-phenylalanyl-glycine, L- or D-tyrosyl-glycine, L- or D-leucyl-glycine, L- or D-phenylalanyl-citrulline and L- or D-valyl-citrulline.

6. The pharmaceutical composition according to claim 1, wherein the polysaccharide having a carboxyl group is a carboxymethylated dextran, the active substance having an anti-tumor activity is 10-(3′-aminopropyloxy)-7-ethyl-(20S)-camptothecin, and the peptide is glycyl-glycyl-glycine.

7. The pharmaceutical composition according to claim 1, wherein the polysaccharide having a carboxyl group is a carboxy-C₁₋₄ alkyldextran polyalcohol, the active substance having an anti-tumor activity is (1S, 9S)-1-amino-9-ethyl-5-fluoro-2,3-dihydro-9-hydroxy-4-methyl-1H,12H-benzo[de]-pyrano[3′,4′:6,7]indolidino[1,2-b]quinoline-10,13(9H,15H)-dione, and the peptide is glycyl-glycyl-L- or D-phenyl-alanyl-glycine.

8. The pharmaceutical composition according to any one of claims 1 to 7, which is a pharmaceutical composition for inhibiting the metastasis of a malignant tumor.

9. The pharmaceutical composition according to any one of claims 1 to 7, which is a pharmaceutical composition for preventing the recurrence of a malignant tumor.
